(19) United States
(12) Patent Application Publication
(10) Pub. No.: US 2026/0360483 A1
(43) Pub. Date: Nov. 12, 2026

(54) ADAPTIVE MACHINE LEARNING BASED CLINICAL TRIAL ELIGIBILITY ASSESSMENT

(71) Applicant: BOARD OF REGENTS, THE UNIVERSITY OF TEXAS SYSTEM, Austin, TX (US)

(72) Inventors: Michael Dohopolski, Irving, TX (US); Ann Marie Navar, Dallas, TX (US)

(73) Assignee: BOARD OF REGENTS, THE UNIVERSITY OF TEXAS SYSTEM, Austin, TX (US)

(21) Appl. No.: 19/672,287

(22) Filed: May 8, 2026

Related U.S. Application Data
(60) Provisional application No. 63/803,339, filed on May 9, 2025.

(51) Int. Cl. G16H 10/20; G16H 50/20

(52) U.S. Cl. CPC G16H 10/20; G16H 50/20

(57) ABSTRACT

This disclosure provides systems, methods, and devices to perform clinical trial eligibility screening using machine learning based on user input and patient data. In one aspect, a method is provided that includes receiving a first user input indicating at least one aspect of a clinical trial. A first request is determined based on the user input, the request specifying one or more conditions required to satisfy a first eligibility requirement for the trial. Based on the first request, one or more relevant data segments for a first patient are determined from a data store comprising patient data. A first machine learning model then determines a first classification of the first patient for the first eligibility requirement based at least in part on the first request and the relevant data segments.

### CROSS REFERENCE TO RELATED APPLICATION

This application claims priority to U.S. Provisional Patent Application Ser. No. 63/803,339, filed May 9, 2025, and titled “ADAPTIVE MACHINE LEARNING BASED CLINICAL TRIAL ELIGIBILITY ASSESSMENT,” which is incorporated by reference herein in its entirety.

### BACKGROUND

### 1. Field

This disclosure pertains generally to the field of clinical trial management, and particularly to systems and methods employing machine learning for automated screening of patient eligibility using electronic health records.

### 2. Discussion of Related Art

Clinical trials may represent a cornerstone of medical research and development, facilitating the evaluation of new therapies, diagnostic methods, and medical devices. The execution of clinical trials often involves identifying and recruiting suitable participants who meet specific criteria outlined in a trial protocol. This process typically relies on accessing and interpreting patient health information.

Electronic Health Records (EHRs) have become widespread in healthcare settings, serving as digital repositories for patient data. EHR systems may store a wide range of information, including demographics, medical history, diagnoses, medications, laboratory results, and clinical notes documented by healthcare providers. This wealth of data holds potential for various application[s beyond direct patient care, including supporting clinical research activities such as identifying potential candidates for clinical trials.

### SUMMARY

The present disclosure relates to techniques for evaluating clinical trial eligibility. A system may receive user input indicating aspects or requirements of a clinical trial. Based on this input, the system may determine a request, potentially involving generating a prompt using machine learning models, querying external data sources, or retrieving stored requests from a library. The system may then determine relevant data segments for a patient from a data store containing patient data, such as electronic health records (EHR), potentially using vector embeddings and similarity searches. A first machine learning model may process the request and relevant segments to determine a classification for the patient regarding an eligibility requirement, possibly including a certainty measure. This process may be repeated for multiple requirements and patients. An overall eligibility classification, potentially indicating a proportion of requirements met, may be determined. Results, including classifications, certainty measures, and eligibility classifications, may be presented via a user interface, potentially including a dashboard for prioritizing patients. The system may also incorporate a feedback loop, receiving reviewer assessments, identifying discrepancies with model classifications, determining adjustments to requests (potentially using another machine learning model), and applying these adjustments to refine future requests, which may be stored back in the request library. Performance metrics may be calculated based on reviewer feedback and presented to users. Machine learning models may also be used to generate structured data queries for initial patient cohort selection.

In a first aspect, a method includes receiving a first user input that indicates at least one aspect of a clinical trial; determining a first request based on the user input, the first request specifying one or more conditions required to satisfy a first eligibility requirement corresponding to the clinical trial; determining, based at least in part on the first request, one or more relevant data segments for a first patient of a plurality of patients from a data store including patient data for at least a subset of the plurality of patients; and determining, based at least in part on the first request and the one or more relevant data segments, a first classification of the first patient for the first eligibility requirement using a first machine learning model.

In a second aspect according to the first aspect, the at least one aspect of the clinical trial indicated by the user input includes the first eligibility requirement.

In a third aspect according to any one of the first through second aspects, the at least one aspect of the clinical trial indicated by the user input includes at least one of a medical condition, a diagnostic category, a therapeutic area, a patient demographic, or a combination thereof.

In a fourth aspect according to any one of the first through third aspects, the first machine learning model is a large language model, and the first request includes a prompt configured for execution by the first machine learning model.

In a fifth aspect according to the fourth aspect, the determining of the first request includes determining, based on the user input, the prompt using the first machine learning model, a second machine learning model, or a combination thereof.

In a sixth aspect according to the fifth aspect, the method further includes determining one or more external data sources based on the user input and querying the one or more external data sources, wherein the determining of the first request is further based on results from the querying of the one or more external data sources.

In a seventh aspect according to the sixth aspect, the one or more external data sources includes at least one of a public medical literature database, a clinical trial registry, or a curated knowledge base.

In an eighth aspect according to any one of the first through seventh aspects, the method further includes determining, by the first machine learning model, a first certainty measure associated with the first classification.

In a ninth aspect according to any one of the first through eighth aspects, the method further includes repeating the step of determining a first classification for each patient of at least a second subset of the plurality of patients.

In a tenth aspect according to any one of the first through ninth aspects, the method further includes determining, based on the first classification for the first patient, an eligibility classification of the first patient for the clinical trial.

In an eleventh aspect according to the tenth aspect, the method further includes repeating the steps of determining the one or more relevant data segments and determining the first classification for each eligibility requirement of a plurality of eligibility requirements corresponding to the clinical trial prior to determining the eligibility classification of the first patient, wherein the eligibility classification is determined based on the classifications determined for the plurality of eligibility requirements.

In a twelfth aspect according to the eleventh aspect, the eligibility classification indicates a proportion of the plurality of eligibility requirements for which the determined classification indicates the requirement is met.

In a thirteenth aspect according to any one of the first through twelfth aspects, the method further includes selecting the plurality of patients based at least in part on the user input, the first request, or a combination thereof prior to the determining of the one or more relevant data segments.

In a fourteenth aspect according to the thirteenth aspect, the selecting of the plurality of patients includes determining, using a machine learning model based on the user input, the first request, or a combination thereof, one or more structured data queries, and executing the one or more structured data queries against a larger patient population including the plurality of patients.

In a fifteenth aspect according to any one of the first through fourteenth aspects, the method further includes receiving a second user input via a user interface, the second user input indicating a reviewer assessment of the first classification for the first patient.

In a sixteenth aspect according to the fifteenth aspect, the method further includes determining that a discrepancy exists between the first classification and the reviewer assessment indicated by the second user input; determining, based at least in part on the discrepancy, an adjustment applicable to the first request based on at least one of the second user input, the first request, or a combination thereof; and determining a second request by applying the adjustment to the first request.

In a seventeenth aspect according to the sixteenth aspect, the method further includes receiving a third user input indicating a recruitment outcome for the first patient in the clinical trial, wherein the adjustment is determined based further on the third user input.

In an eighteenth aspect, a system includes a processor and a memory storing instructions which, when executed by the processor, cause the processor to perform operations including receiving a user input that indicates at least one aspect of a clinical trial; determining a first request based on the user input, the first request specifying one or more conditions required to satisfy a first eligibility requirement corresponding to the clinical trial; determining, based at least in part on the first request, one or more relevant data segments for a first patient of a plurality of patients from a data store including patient data for at least a subset of the plurality of patients; and determining, based at least in part on the first request and the one or more relevant data segments, a first classification of the first patient for the first eligibility requirement using a first machine learning model.

In a nineteenth aspect according to the eighteenth aspect, the operations further include receiving a second user input via a user interface, the second user input indicating a reviewer assessment of the first classification for the first patient; determining that a discrepancy exists between the first classification and the reviewer assessment indicated by the second user input; determining, based at least in part on the discrepancy, an adjustment applicable to the first request based on at least one of the second user input, the first request, or a combination thereof; and determining a second request by applying the adjustment to the first request.

In a twentieth aspect, a non-transitory, computer-readable medium stores instructions which, when executed by a processor, cause the processor to perform operations including receiving a user input that indicates at least one aspect of a clinical trial; determining a first request based on the user input, the first request specifying one or more conditions required to satisfy a first eligibility requirement corresponding to the clinical trial; determining, based at least in part on the first request, one or more relevant data segments for a first patient of a plurality of patients from a data store including patient data for at least a subset of the plurality of patients; and determining, based at least in part on the first request and the one or more relevant data segments, a first classification of the first patient for the first eligibility requirement using a first machine learning model.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the disclosed subject matter.

### BRIEF DESCRIPTION OF THE FIGURES

FIG. 1 illustrates a system for clinical trial eligibility evaluation according to one aspect of the present disclosure.

FIGS. 2A-2D illustrate user interfaces for clinical trial eligibility evaluation according to aspects of the present disclosure.

FIG. 3 illustrates a method for clinical trial eligibility evaluation according to one aspect of the present disclosure.

FIG. 4 illustrates a computing system according to one aspect of the present disclosure.

### DETAILED DESCRIPTION

Identifying and screening patients for clinical trial eligibility may present significant challenges. Traditional methods often involve manual review of patient charts and records by clinical research staff. This manual screening process may be extremely time-consuming and labor-intensive, potentially requiring substantial effort per patient to determine if they meet the often complex inclusion and exclusion criteria specified in a trial protocol. The reliance on manual review may also lead to inconsistencies, potential errors in interpretation, and limitations in the scale and speed at which patients can be screened. Consequently, clinical trial recruitment may be slow, costly, and inefficient, sometimes leading to trial delays or failures due to insufficient enrollment.

Prior attempts to automate patient screening have included rule-based systems and techniques using traditional machine learning or natural language processing (NLP). Rule-based systems may attempt to match predefined keywords or patterns corresponding to eligibility criteria within patient data. However, these systems may be brittle, requiring significant domain expertise to create and maintain the rules. They may also struggle to interpret the nuances, context, and temporal relationships often present in unstructured clinical text, potentially leading to inaccurate assessments. Structured data within electronic health records (EHRs), while useful, may often be insufficient to capture the full complexity of many eligibility criteria, much of which may only be documented in free-text notes. Traditional NLP and machine learning approaches may require extensive data preprocessing, feature engineering, and the availability of large, labeled datasets for training, which may limit their adaptability and scalability across diverse clinical trials and criteria. These methods may also face difficulties in deeply understanding the semantic content of unstructured clinical narratives.

One solution to this problem is to employ techniques that leverage advanced machine learning models, such as Large Language Models (LLMs), combined with data retrieval methods to analyze patient data, including unstructured text within electronic health records (EHRs). The described techniques may involve receiving user input defining aspects or requirements of a trial and determining a corresponding request, such as formatted as a prompt suitable for an LLM. This request determination may itself utilize machine learning, access external data sources, or query a library of stored requests, allowing for flexibility and adaptation to various criteria. To handle potentially large volumes of patient data vector embeddings may be created from patient data portions and performing similarity searches to identify the most relevant data segments for a given request.

A first machine learning model may then analyze these relevant segments based on the request to produce a classification regarding an eligibility requirement, potentially indicating states like ‘Met’, ‘Not Met’, or ‘Uncertain’, along with a certainty measure. This addresses the limitations of rule-based systems in handling nuance and context. By assessing multiple requirements and aggregating the results, an overall eligibility classification may be determined, potentially as a proportion of criteria met, offering a more practical assessment than strict all-or-nothing matching. Furthermore, the techniques may incorporate a feedback loop. Reviewer assessments of the model's classifications may be received via a user interface. Discrepancies between the model's output and the reviewer's assessment may trigger the determination of an adjustment, potentially using another machine learning model, which is then applied to refine the initial request into an improved second request. This refined request may be used for subsequent patients and stored back in the request library, allowing the system to adapt and improve its accuracy over time based on expert feedback, addressing the static nature of prior systems. Machine learning may also be used to generate structured data queries for initial filtering of large patient populations, improving efficiency.

In some aspects, the present disclosure provides techniques for automating clinical trial eligibility assessment that yield significant practical benefits. By utilizing machine learning models, particularly LLMs, integrated with specific data processing techniques to analyze unstructured EHR data directly, the described system significantly reduces the time and manual effort required for patient screening compared to existing automated methods like rule-based systems or traditional NLP. In particular, the described techniques offer greater flexibility in handling diverse and complex eligibility criteria due to the semantic understanding capabilities of LLMs and the adaptive nature of the request generation. This technical implementation improves the computer's functionality through several mechanisms. The RAG technique, involving vector embedding generation, indexing, and similarity searching, provides a computationally efficient method for querying and retrieving relevant information from large volumes of unstructured EHR text, optimizing resource utilization. The integrated adaptive feedback loop represents a concrete improvement in the computer's operation; by computationally analyzing discrepancies, determining adjustments based on reviewer feedback, and automatically generating refined machine-executable requests, the system adaptively improves its core processing logic over time without requiring manual recoding, enhancing accuracy and reliability. The overall workflow optimizes computational analysis by concentrating machine learning model processing on the most pertinent data, and the automated generation of structured database queries further improves the efficiency of initial patient cohort filtering.

These technical improvements directly enable concrete, practical advantages in the clinical research domain and enhance the experience for end users like clinical research staff. The system's ability to computationally analyze nuances in clinical text, coupled with the adaptive request refinement mechanism, results in more accurate and consistent eligibility assessments than previous automated systems, specifically addressing the challenge of criteria hidden in narrative text. The techniques allow the system to be adapted to new trials and criteria with potentially less effort than systems requiring extensive manual rule creation or specialized model training. Additionally, the system generates specific, practical outputs integrated into clinical workflow tools (user interface/dashboard), such as prioritized candidate lists ranked by quantitative eligibility scores, individual classifications with certainty measures, and performance metrics. This provides actionable intelligence, enabling staff to allocate resources more effectively. The effective handling of real-world unstructured data complexity, combined with increased transparency through displayed certainty and performance metrics, fosters informed decision-making and user trust in the automated results within the high-stakes clinical environment. The feedback mechanism enables users to contribute to the system's ongoing improvement, further building confidence in its outputs and improving model transparency.

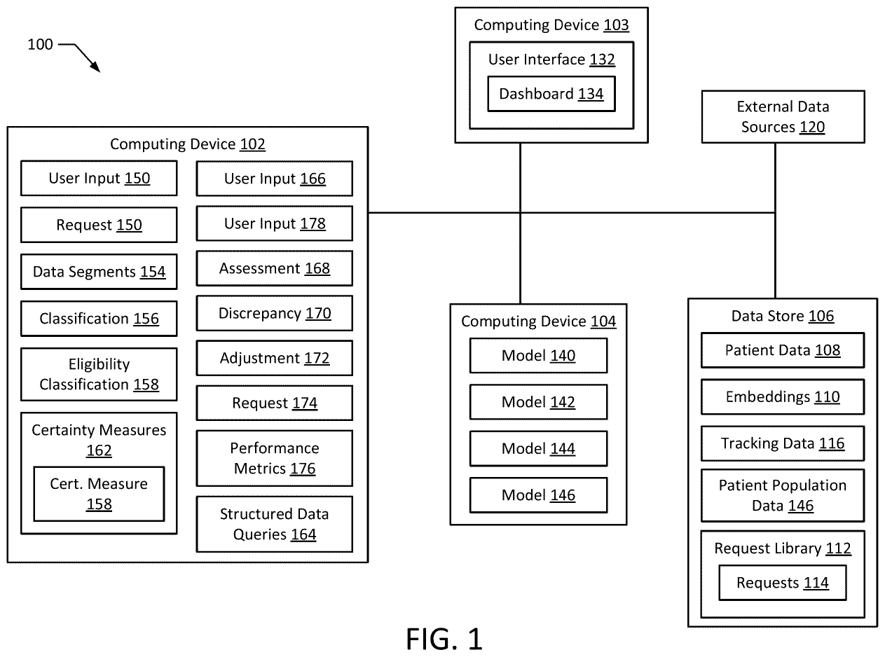

FIG. 1 depicts a system 100 for clinical trial eligibility evaluation according to one aspect of the present disclosure. The system 100 may include a first computing device 102, a second computing device 104, and a third computing device 103. The first computing device 102 may process or store various data elements, including user input 150, a first request 152, relevant data segments 154, a first classification 156, a first certainty measure 158, an eligibility classification 160, certainty measures 162, structured data queries 164, a second user input 166 (which may indicate a reviewer assessment 168), discrepancy information 170, an adjustment 172, a second request 174, performance metrics 176, and a third user input 178. The third computing device 103 may host or interact with a user interface 132, which may include a dashboard 134. The second computing device 104 may host or interact with various machine learning models, including a first machine learning model 140, a second machine learning model 142, a third machine learning model 144, and a fourth machine learning model 146. The system 100 may also include or interact with a data store 106, which may contain patient data 108 (including EHR data), vector embeddings 110, a request library 112 (containing stored requests 114), tracking data 116, and larger patient population data 118. The system 100 may further interact with external data sources 120.

In certain implementations, the system 100 architecture may include the first computing device 102 communicatively coupled to a second computing device 104 and potentially a third computing device 103. For example, the first computing device 102 may host a user interface 132 accessible via the third computing device 103 (acting as a client terminal), while the second computing device 104 performs backend processing, such as executing machine learning models 140, 142, 144, 146. Communication pathways between these devices 102, 103, 104 may utilize standard network protocols, such as HTTPS over TCP/IP, ensuring secure data transmission. The user input 150 may be received by the first computing device 102 through various mechanisms presented via the user interface 132 on the third computing device 103. For instance, the user interface 132 may include a text entry field allowing free-text input, selectable options presented via dropdown menus, checkboxes, mechanisms for uploading documents (such as a clinical trial protocol document) that contain relevant aspects or criteria, or a combination thereof. The first computing device 102 may be configured to parse and interpret the received user input 150 to extract the indicated aspect(s). This parsing may involve natural language processing techniques if the input is free text or simple extraction if structured input elements are used. For example, a user interface 132 might present fields for “Trial Name/ID,” “Condition,” “Intervention,” and “Key Eligibility Criteria,” allowing the user to input various aspects defining the scope of the screening task.

The computing device 102 may be configured to receive a user input 150 that indicates at least one aspect of a clinical trial. In certain configurations, the at least one aspect of the clinical trial indicated by the user input 150 received by the first computing device 102 may directly include the first eligibility requirement. An eligibility requirement may refer to a specific condition or characteristic that a patient must meet (or not meet) to qualify for participation in the clinical trial. For instance, a user might directly input a specific, fully formed eligibility requirement such as “ECOG performance status 0-2” or “HbA1c between 6.5% and 9.5%” into a designated field in the user interface 132. In such scenarios, the received user input 150 itself may constitute the definition of the requirement.

In other configurations, the at least one aspect of the clinical trial indicated by the user input 150 received by the first computing device 102 may include at least one of a medical condition, a diagnostic category, a therapeutic area, a patient demographic, or a combination thereof. A medical condition may refer to a specific disease, disorder, or state relevant to the trial, for example, ‘Stage IV Prostate Cancer’ or ‘Type 2 Diabetes Mellitus’. A diagnostic category may refer to a broader classification of diseases or conditions, such as ‘Oncology’, ‘Infectious Diseases’, or ‘Autoimmune Disorders’. A therapeutic area may refer to a field of medicine the trial pertains to, for instance, ‘Cardiology’, ‘Neurology’, or ‘Pulmonology’. A patient demographic may refer to characteristics of the target patient population, such as ‘Age>=18 years’, ‘Female’, or ‘History of Smoking’. Receiving these broader aspects may guide subsequent operations performed by the first computing device 102 or the second computing device 104, such as searching a request library 112 for relevant stored requests 114 or tailoring the determination of the first request 152 to focus on eligibility requirements commonly associated with the specified aspect.

In further configurations, the user input 150 received by the first computing device 102 may indicate other types of aspects related to the clinical trial. These aspects may include logistical or administrative details, such as the trial phase (such as ‘Phase II’, ‘Phase III’), a specific study identifier (such as ‘NCT12345678’), the name of the principal investigator, or a combination thereof. The first computing device 102 may be configured to differentiate between receiving a specific, actionable eligibility requirement versus receiving a broader aspect. For example, inputs recognized as specific criteria might directly lead to the determination of a corresponding request 152, while broader aspects might trigger intermediate operations, such as querying the request library 112 or invoking a machine learning model 142 to help formulate potential eligibility requirements and requests based on the provided aspect.

The first computing device 102 may also be configured to determine a first request 152 based on the received user input 150. The first request 152 may specify one or more conditions required to satisfy a first eligibility requirement corresponding to the clinical trial. A request may refer to a structured or unstructured query, prompt, set of instructions used to guide data retrieval or model execution, or a combination thereof. Conditions may refer to the specific logical, textual, or numerical constraints defining the eligibility requirement. This determination operation may include translating the potentially broad user input ‘aspect’ into a more concrete representation suitable for guiding subsequent data retrieval and analysis, and explicitly linking the request 152 to a specific eligibility requirement being assessed. The format and structure of the first request 152 may vary. For example, the first computing device 102 may determine the first request 152 to be represented as structured data (such as key-value pairs defining parameters), a natural language prompt designed for a machine learning model, a set of logical rules (such as Boolean expressions), or a combination thereof. The conditions specified within the first request 152 represent the specific details or logic needed to evaluate the corresponding eligibility requirement. For instance, if the user input 150 indicates the aspect ‘Hypertension Trial’, the first computing device 102 might determine a first request 152 corresponding to the eligibility requirement ‘Blood Pressure Control’, specifying conditions such as ‘Systolic BP<140 mmHg AND Diastolic BP<90 mmHg’. As another example, if the user input 150 is the specific criterion ‘ECOG<=2’, the first computing device 102 may determine the first request 152 to directly encode the condition ‘ECOG performance status must be 0, 1, or 2’.

In certain implementations, the first request 152 determined by the first computing device 102 may include a prompt configured for execution by the first machine learning model 140. A prompt may refer to the structured text input provided to a machine learning model 140 to guide the model's operation. Various prompt structures may be suitable for machine learning models 140 in the context of clinical trial screening. Examples include zero-shot prompts (providing instructions without specific examples), few-shot prompts (providing instructions along with a small number of examples), structured output prompts (instructing the model 140 to format its output in a specific way, such as JSON), Chain-of-Thought (CoT) prompts (instructing the model 140 to articulate its reasoning steps before providing a final answer), prompts utilizing frameworks like Self-Discover (allowing the model 140 to autonomously construct a reasoning process), or a combination thereof. Prompt templates may be employed by the first computing device 102, incorporating placeholders for the specific conditions derived from the user input 150. For example, a template might be: “Based on the provided text, determine if the patient meets the criterion: ‘[Condition specified in first request]’. Provide your classification (Met/Not Met/Uncertain) and supporting evidence.”

In some implementations, the first computing device 102 may determine the first request 152, potentially including the prompt, using the first machine learning model 140, a second machine learning model 142, or a combination thereof, based on the user input 150. A second machine learning model 142, potentially distinct from the first machine learning model 140 used for classification and potentially hosted on the second computing device 104, may be dedicated to automatically generating the prompt text based on the received user input ‘aspect’. The input to this prompt-generating model 142 may be the user input text 150, and its output may be the formatted prompt text (the first request 152). Alternatively, the first machine learning model 140 itself might be used by the first computing device 102 or second computing device 104 in a preliminary step to refine or generate the prompt based on the initial user input 150. For example, a user might input “T4 prostate cancer,” and the second machine learning model 142 may generate a detailed prompt incorporating conditions related to tumor invasion specified in AJCC staging, which may then be determined as the first request 152, ready for use by the first machine learning model 140.

The process of determining the prompt via a machine learning model 142 may be further enhanced by incorporating external information. The first computing device 102 or second computing device 104, such as guided by the second machine learning model 142, may determine one or more relevant external data sources 120 based on the user input 150. For instance, keywords like ‘cancer staging’ or specific drug names in the user input 150 might be used to identify sources like medical literature databases or drug information databases. The computing device 102, 104 may then query the identified external data sources 120, which may involve formulating search queries (such as PubMed search terms) or making API calls to the external source 120. For example, upon receiving the user input ‘T4 prostate cancer’, the second machine learning model 142 might generate search terms like “T4 prostate cancer definition AJCC 8th edition” and execute a programmatic search against the PubMed database via its API. The first computing device 102 may then determine the first request 152 based further on the results obtained from querying the one or more external data sources 120. The query results, such as text snippets containing definitions, classification criteria, or relevant context, may be incorporated into the first request 152 or prompt to make the request more informative and accurate. For example, if the user input 150 is ‘T4 prostate cancer’, the second machine learning model 142 might query PubMed as described, retrieve AJCC staging definitions for T4 prostate cancer, and the first computing device 102 may integrate the retrieved definitions as specific conditions or ‘tips’ within the generated prompt (first request 152).

The one or more external data sources 120 may include public medical literature databases, clinical trial registries, curated knowledge bases, or a combination thereof. Public medical literature databases may refer to repositories of biomedical articles and abstracts, such as PubMed or MEDLINE. Clinical trial registries may refer to databases listing clinical studies, such as ClinicalTrials. gov. Curated knowledge bases may refer to structured repositories of biomedical information, such as the Unified Medical Language System (UMLS), Systematized Nomenclature of Medicine-Clinical Terms (SNOMED-CT), drug information databases like RxNorm, or a combination thereof. Information retrieved from these sources 120, such as standard definitions, inclusion/exclusion criteria examples from similar trials, or structured concept relationships, can significantly aid the first computing device 102 in determining a precise and effective first request 152.

In additional or alternative implementations, the first computing device 102 may determine the first request 152 by querying a request library 112. The first computing device 102 may query a request library 112 storing a plurality of stored requests 114 associated with a plurality of eligibility requirements, based on the user input 150. The request library 112 may be organized as a database or structured file system residing within the data store 106. Stored requests 114 may be indexed or categorized based on various attributes, such as the associated clinical trial, the type of eligibility requirement (such as diagnosis, lab value, prior treatment), or underlying medical concepts (such as mapped to SNOMED-CT codes). The querying performed by the first computing device 102 may involve keyword searching based on the user input 150, natural language querying, vector-based semantic search techniques (potentially using vector embeddings of the user input 150 and the stored requests 114, similar to Retrieval-Augmented Generation or RAG), structured metadata querying, or a combination thereof. For example, if the user inputs ‘ECOG performance status’, the device 102 might perform a semantic search in the library 112 to find previously validated prompts 114 related to assessing ECOG status. At least one stored request 114 may be retrieved by the first computing device 102 from the request library 112 based on the querying. The first computing device 102 may then determine the first request 152 based at least in part on the retrieved at least one stored request 114. For example, a retrieved stored request 114 deemed highly relevant based on the user input 150 (e.g., a previously used prompt for ‘ECOG<=2’ with high performance metrics) might be used directly as the first request 152 if the current requirement is identical. Alternatively, the first computing device 102 might adapt the retrieved request, modify specific parameters within the request, merge the retrieved request 114 with information derived directly from the user input 150 or external sources 120, or perform a combination thereof, to determine the final first request 152. For instance, if the user input indicates ‘prior radiotherapy exclusion’ for a head and neck cancer trial, the first computing device 102 might retrieve a validated stored request 114 for a similar exclusion from a previous trial and adapt the retrieved request slightly for the current context, perhaps by adjusting anatomical keywords based on the specifics of the current trial protocol.

Alternative methods for determining the first request 152 may also be employed by the first computing device 102. For example, rule-based systems may be configured to translate specific keywords or patterns in the user input 150 into predefined request structures. Direct mapping from selected criteria templates presented in the user interface 132 may also be used to determine the first request 152.

The first computing device 102 may also be configured to determine, based at least in part on the first request 152, one or more relevant data segments 154 for a first patient of a plurality of patients from a data store 106 including patient data 108 for at least a subset of the plurality of patients. The data store 106 may be implemented using various database technologies (such as relational databases (e.g., PostgreSQL®, SQL Server®), NoSQL databases (e.g., MongoDB®, Cassandra®), vector databases (e.g., Pinecone®), data warehouses, data lakes, or a combination thereof) and may be located locally on the first computing device 102, second computing device 104, or hosted remotely, such as in a cloud computing environment (e.g., AWS, Google Cloud, Azure). Appropriate security measures, potentially including encryption at rest and in transit, access controls (e.g., role-based access control), audit logging, network segmentation, vulnerability management, and data masking/pseudonymization where appropriate, may be implemented to protect the patient data 108, ensuring compliance with regulations such as HIPAA, GDPR, or other applicable data privacy laws. Patient data 108 within the data store 106 may be organized in various ways, such as using patient-specific document collections in a NoSQL database, relational tables linked by patient identifiers, dedicated schemas per patient, or indices mapping patient IDs to relevant data pointers, to facilitate efficient retrieval of data pertaining to a particular individual. The first patient may then be selected sequentially or based on prioritization from the plurality of patients, which may represent an initial selected cohort.

In certain implementations, the patient data 108 in the data store 106 may include electronic health record (EHR) data. EHR data may refer to the digital records of patient health information. EHR data may be received from one or more EHR systems, such as systems provided by Epic, Cerner, and the like. The first computing device 102 or an associated data ingestion pipeline may be configured to extract various types of data from these EHR sources, potentially including structured data such as laboratory results, codified problem lists (such as using ICD codes), medication lists (such as using RxNorm® codes), and patient demographics, as well as unstructured data such as free-text clinical notes, pathology reports, radiology or other imaging reports. Furthermore, the EHR data within the patient data 108 may include unstructured text data. Unstructured text data may refer to free-form narrative text that does not adhere to a predefined data model. For example the unstructured text data may include History and Physical (H&P) notes, physician consult notes, progress notes, discharge summaries, operative reports detailing surgical procedures, or the findings or impression sections of radiology reports (such as CT, MRI, PET scans).

In certain configurations, the first computing device 102 may determine the one or more relevant data segments 154 by first determining a plurality of vector embeddings 110 corresponding to data portions within the patient data 108 for the first patient. The first computing device 102 may then determine the one or more relevant data segments 154 by performing a similarity search over the plurality of vector embeddings 110 based on the first request 152. Creating vector embeddings 110 may involve document chunking, where larger documents (like clinical notes) are divided into smaller, manageable data portions. Chunking strategies may include using a fixed token size (such as 1024 tokens), splitting by sentences, splitting by paragraphs, dividing based on document sections (such as ‘Assessment and Plan’, ‘Past Medical History’), or using recursive character splitting. In certain implementations, an overlap between chunks (such as an overlap of 100 tokens for 1024-token chunks) may be used to preserve contextual continuity. A text embedding model may then be used by the first computing device 102 or second computing device 104 to convert each data portion into a high-dimensional numerical vector (embedding). The dimensionality of the resulting vectors may vary depending on the model used (e.g., 1536 dimensions for text-embedding-ada-002). The resulting vector embeddings 110 may be stored, potentially in a specialized vector database technology (such as Pinecone®, FAISS, Weaviate®, Milvus®, Qdrant®, or a combination thereof) within the data store 106, indexed for efficient searching. The similarity search performed by the first computing device 102 may include comparing an embedding of the first request 152 (or parts thereof) to the stored vector embeddings 110 using a distance metric, such as cosine similarity or Euclidean distance (e.g., L2 distance). Algorithms such as k-Nearest Neighbors (k-NN) or Approximate Nearest Neighbors (ANN) techniques may be employed to find the ‘k’ vector embeddings 110 closest (most similar) to the request embedding. The corresponding original data portions (segments) may then be retrieved as the relevant data segments 154. In certain implementations, the value for ‘k’ may have a value between 3 and 10 (such as 5), although other implementations may differ. In certain implementations, frameworks such as LlamaIndex or LangChain may be used by the first computing device 102 to orchestrate the RAG process, including chunking, embedding, indexing, and retrieval. In additional or alternative implementations, the computing device 102 may be configured to utilize different techniques for determining relevant segments, such as keyword searching (such as using ElasticSearch or OpenSearch), applying predefined rules or regular expressions to extract specific patterns, or employing topic modeling to identify documents or sections relevant to the concepts in the first request 152.

In certain implementations, the first computing device 102 may be configured to select the plurality of patients based at least in part on the user input 150, the first request 152, or a combination thereof, prior to determining the one or more relevant data segments 154. This initial filtering or cohort selection may narrow down a larger patient population to a more manageable group for the detailed, computationally intensive screening process involving the first machine learning model 140. Data sources for this filtering may include the same or similar data sources included in or accessible by the data store 106 and/or the external data sources 120. In certain implementations, the filtering logic applied by the first computing device 102 may be determined based on the user input 150. For example, if the user input 150 indicates a ‘prostate cancer’ trial, the first computing device 102 might apply a filter to select only male patients from the larger patient population data 118. Similarly, specific diagnostic codes, age ranges, or lab value thresholds mentioned or implied might be used for initial filtering.

The selection of the plurality of patients may involve the use of a machine learning model, such as one of the models 142, 144, 146. The first computing device 102 may be configured such that selecting the plurality of patients includes determining, using a third machine learning model 146 based on the user input 150, the first request 152, or a combination thereof, one or more structured data queries 164. A structured data query 164 may refer to a query formulated in a language designed for retrieving data from structured databases, such as SQL (Structured Query Language) or SPARQL for RDF databases. The third machine learning model 146, which may be implemented as an LLM trained or prompted for query generation, may translate the natural language or semi-structured information from the user input 150 or first request 152 into formally correct structured data queries 164. For example, the third machine learning model 146 might translate the requirement “ECOG<=2” into an SQL query targeting a specific table and column storing ECOG performance status values in an EHR database. The first computing device 102 may then execute the one or more determined structured data queries 164 against the larger patient population data 118 within the data store 106 to retrieve the list of patients forming the selected plurality of patients.

Subsequently, the first computing device 102 may be configured to determine, based at least in part on the first request 152 and the one or more relevant data segments 154, a first classification 156 of the first patient for the first eligibility requirement using a first machine learning model 140. The inputs to the first machine learning model 140, potentially hosted on or accessed via the second computing device 104, typically include the one or more relevant data segments 154 determined in the previous step and the first request 152 (often formatted as a prompt). The first machine learning model 140 processes these inputs, analyzing the text of the relevant data segments 154 in the context of the conditions specified in the first request 152, to determine the first classification 156.

In some implementations, the first classification 156 determined by the first machine learning model 140 may indicate whether the first patient meets the first eligibility requirement, such as a binary classification. For instance, the first machine learning model 140 may be configured or prompted to output a simple ‘Met’ or ‘Not Met’ label, or an equivalent representation (such as True/False, 1/0). In other implementations, the first classification 156 determined by the first machine learning model 140 may indicate a non-binary status, such as a status selected from: Met, Not Met, Likely Met, Likely Not Met, and Uncertain. This outcome represents a multi-category classification, offering more nuance than a simple binary outcome. ‘Met’ and ‘Not Met’ may indicate higher confidence in the assessment based on clear evidence. ‘Likely Met’ and ‘Likely Not Met’ may indicate a probable status based on suggestive but not definitive evidence, or perhaps lower model confidence. ‘Uncertain’ may indicate insufficient or contradictory evidence, or the model's inability to make a determination. The first machine learning model 140 may distinguish between these levels based on internal confidence thresholds applied to its output probabilities and/or based on specific patterns or lack thereof identified in the evidence. The model's output format may be a categorical label.

The first computing device 102 may further determine, potentially via the first machine learning model 140, a first certainty measure 158 associated with the first classification 156. A certainty measure may refer to a quantifiable score indicating the model's confidence in its determined classification. This measure may be generated directly by the first machine learning model 140, for example, as a probability score associated with the predicted class (such as the softmax output for the chosen classification label) or derived from internal model states such as attention weights. The certainty measure 158 might be represented on a numeric scale, such as 0 to 1 or as a percentage. This measure can be used downstream, for instance, to weight the classification when calculating a probabilistic eligibility score, to prioritize patients or criteria for human review (such as reviewing classifications with low certainty first), or as an input to the secondary model (if used) for determining overall eligibility. For example, a classification of ‘Met’ with a certainty measure of 0.95 might be treated differently in subsequent steps than a classification of ‘Met’ with a certainty measure of 0.60.

The first computing device 102 may further be configured to repeat the step of determining a first classification 156 for each patient of at least a second subset of the plurality of patients. In particular, the second subset of the plurality of patients may represent a cohort of candidate patients for the clinical trial, and the computing device 102 may be configured to determine a first classification 156 for each of these patients.

In implementations evaluating multiple requirements, the first computing device 102 may be configured to repeat the steps of determining one or more relevant data segments 154 and determining a first classification 156 for each eligibility requirement of a plurality of eligibility requirements corresponding to the clinical trial prior to determining the eligibility classification 160 of the first patient. In particular, the computing device 102 may be configured to separately analyze each criterion, or each subset of criterion against corresponding patient information on a per-patient basis. The eligibility classification 160 for the patient may then be determined by the first computing device 102 based on the aggregation or synthesis of the individual classifications 156 determined for the plurality of eligibility requirements. For example, a trial might have ten distinct eligibility requirements, and the first computing device 102 may be configured to determine ten corresponding classifications 156 for the patient. In additional or alternative implementations, the first computing device 102 may be configured such that determining the first certainty measure 158 includes determining a certainty measure 158 associated with the first classification 156 for each of the plurality of eligibility requirements.

In some configurations, the first computing device 102 may be configured to determine, based on the first classification 156 for the first patient, an eligibility classification 160 of the first patient for the clinical trial. 25. In certain implementations, the first computing device 102 may be configured to determine the eligibility classification 160 for the first patient using the first machine learning model 142, a second machine learning model 144, or a combination thereof. An eligibility classification 160 may refer to an overall assessment of the patient's suitability for the trial, derived from the assessment of one or more specific eligibility requirements. In implementations with multiple eligibility requirements, the computing device 102 may be configured to determine the eligibility classification 160 to indicate a proportion of the plurality of eligibility requirements for which the determined first classification 156 indicates the requirement is met. This indication may represent a proportional eligibility score. The first computing device 102 may calculate this score, for example, by summing the number of criteria classified as ‘Met’ (and potentially ‘Likely Met’, perhaps with a lower weight) and dividing by the total number of eligibility requirements assessed for that patient. For instance, if a patient meets 8 out of 10 criteria, the proportional eligibility classification 160 might be 0.8 or 80%. Such a score provides a quantitative measure useful for ranking patients in terms of likely eligibility and prioritizing them for further review.

In additional or alternative implementations, the computing device 102 may be configured to determine the eligibility classification 160 determined by the first computing device 102 based on whether all of one or more eligibility requirements were met. For example, the first computing device 102 may calculate the computing device may determine at least a portion of the eligibility classification 160 by performing a logical AND operation across the classifications 156 for all mandatory eligibility requirements. In such instances, a patient the eligibility classification 160 of ‘Eligible’ may only be determined if every required criterion's corresponding classification 156 is ‘Met’.

The first computing device 102 may also be configured to present the first classification 156 via a user interface 132. In certain implementations, the user interface 132 may be provided by the first computing device 102, or another computing device, and accessed by users via a third computing device 103. The user interface 132 may include components specifically designed for displaying the results of the individual criterion screening. For example, results for a patient might be shown in a table where each row corresponds to an eligibility requirement, displaying the criterion name and its determined classification 156 (such as a text label ‘Met’, ‘Not Met’, ‘Uncertain’, potentially using color-coding like green for ‘Met’, red for ‘Not Met’). Where a certainty measure is determined, the first computing device 102 may be configured to present the first classification 156 and the first certainty measure 158 via the user interface 132. The certainty measure 158 could be displayed numerically (such as “0.85”, “95%”), visually as a confidence bar or gauge, or textually (such as “High Confidence”, “Low Confidence”) alongside the corresponding classification 156 within the results display. Furthermore, the first computing device 102 may be configured to present the overall eligibility classification 160 via the user interface 132. For example, a patient list could show each patient's name, key demographics, and their overall eligibility score (such as “80%”, “Likely Eligible (0.92)”, “Ineligible (Strict)”), allowing for quick assessment and sorting.

In further implementations, the first computing device 102 may be configured to receive a second user input 166 via a user interface 132, where the second user input 166 indicates a reviewer assessment 168 of the first classification 156 for the first patient. A reviewer assessment 168 may refer to the judgment made by a human expert (such as a clinical research coordinator, physician) regarding the correctness of the classification 156 determined by the first machine learning model 140 for a specific eligibility requirement. The user interface 132, potentially hosted on the first computing device 102 and accessed via a third computing device 103, may present the model's first classification 156 and associated evidence or certainty measure 158. The interface 132 may include elements for capturing the reviewer assessment 168, such as radio buttons labeled ‘Agree’/‘Disagree’ or ‘Correct’/‘Incorrect’, or options allowing the reviewer to select the correct classification (such as ‘Met’, ‘Not Met’). Additionally, the user interface 132 may include a comment box or text field enabling the reviewer to provide free-text explanations or justifications for their assessment 168, especially in cases of disagreement.

In certain implementations, the first computing device 102 may be further configured to determine that a discrepancy 170 exists between the first classification 156 (determined by the first machine learning model 140) and the reviewer assessment 168 indicated by the second user input 166. The first computing device 102 may also determine, based at least in part on the discrepancy 170, an adjustment 172 applicable to the first request 152, where the adjustment 172 determination may be based on at least one of the second user input 166 (including the reviewer assessment 168 and any comments), the first request 152 itself, or a combination thereof. Furthermore, the first computing device 102 may determine a second request 174 by applying the determined adjustment 172 to the first request 152. Detecting the discrepancy 170 may involve comparing the first machine learning model's output (first classification 156) and the reviewer's input (reviewer assessment 168). In certain implementations, the first computing device 102 may be configured to determine the adjustment 172 at least in part using a machine learning model, such as the second machine learning model 142 configured for request analysis and refinement. For example, if the first classification 156 for the ‘No prior head/neck radiotherapy’ requirement was ‘Met’, but the reviewer assessment 168 was ‘Not Met’ with a comment “Patient received radiotherapy for breast cancer only, not head/neck”, the first computing device 102 might provide the first request 152 (e.g., the prompt text including tips like “Look for ‘radiotherapy’, ‘radiation’, ‘XRT’, ‘Gy’”), the discrepancy type (‘False Positive’), and the reviewer's comment to the second machine learning model 142 as input. In certain implementations, the second machine learning model 142 may be configured to process details regarding the discrepancy 170 (such as false positive, false negative), the content of the reviewer's comments within the second user input 166, and the structure or content (such as specific conditions or tips) specified in the first request 152 to determine a predicted cause of the error and formulate an appropriate adjustment 172. For instance, using the previous example, the second machine learning model 142 may analyze that the first request 152 lacked sufficient anatomical specificity based on the reviewer's comment indicating radiation to a different site (breast), predicting this lack of specificity as the cause of the false positive error. An adjustment 172 may refer to a modification applied to the first request 152 intended to correct the identified error. The adjustment 172 may involve actions such as modifying specific keywords or phrases within the prompt, changing the logical structure of conditions, adding constraints or clarifications, or adjusting weighting factors if applicable. Continuing the example, based on identifying the lack of anatomical specificity, the second machine learning model 142 may formulate an adjustment 172 that involves adding keywords related to head and neck anatomy (e.g., ‘larynx’, ‘pharynx’, ‘oral cavity’, ‘neck nodes’) to the ‘tips’ section of the request or adding an explicit condition like ‘Confirm radiation target region includes head or neck’. The first computing device 102 may be configured to apply the determined adjustment 172 to the first request 152 to generate the second request 174, resulting in a refined prompt that guides the first machine learning model 140 to more accurately assess the location of prior radiotherapy.

The second request 174 may then be used for future classifications performed by the first computing device 102. For example, the first computing device 102 may be configured to determine, for a second patient of the plurality of patients, a second classification for the first eligibility requirement using the first machine learning model 140 based on the second request 174 and one or more relevant data segments 154 determined for the second patient. In this way, the computing device 102 may provide an iterative improvement loop where the refined second request 174, generated based on feedback from reviewing the first patient, is then used by the first computing device 102 and first machine learning model 140 during the screening process for subsequent patients (or if the first patient is re-screened). This feedback mechanism allows the system 100 to automatically adapt and improve the accuracy of its requests and subsequent classifications over time as more reviewer feedback 166 is incorporated.

In certain implementations, the first computing device 102 may be further configured to store the second request 174 in a request library 112 associated with the first eligibility requirement. The request library 112, residing in the data store 106, serves as a repository for validated and improved requests. When a refined second request 174 is generated through the feedback loop, the first computing device 102 may save the second request 174 back to the library 112, potentially overwriting the previous version (first request 152) or saving the second request 174 as a new version associated with the same eligibility requirement. Metadata may be stored alongside the request 174 in the library 112, such as the date of the last refinement, associated performance metrics 176 achieved with the request, or a validation status indicating the level of confidence in the request's accuracy. The storage facilitates knowledge management and reuse, as described previously.

In certain implementations, the first computing device 102 may be further configured to receive a third user input 178 indicating a recruitment outcome for the first patient in the clinical trial, wherein the adjustment 172 is determined based further on the third user input 178. A recruitment outcome may refer to the final status of the patient relative to enrollment in the trial, captured potentially at a later time. Examples of recruitment outcomes indicated by the third user input 178 may include ‘Successfully Enrolled’, ‘Screen Failed (Eligibility)’, ‘Screen Failed (Other Reason)’, ‘Declined Participation’, or ‘Completed Trial’. The longer-term outcome information, possibly stored as tracking data 116, can provide valuable signal for refining the screening process. The first computing device 102 may be configured to incorporate the third user input 178 into the analysis when determining the adjustment 172. For example, if multiple patients initially classified as ‘Likely Met’ with moderate certainty consistently fail screening later due to not meeting a specific requirement, the adjustment 172 may be configured to modify the corresponding request 152 to be more detailed an/or strict, may adjust the certainty thresholds associated with a corresponding criterion, and the like.

Based on the received second user input 166, the third user input 178, or a combination thereof, the first computing device 102 may be configured to determine one or more performance metrics 176 associated with the first machine learning model 140 for the first eligibility requirement. Performance metrics 176 may refer to quantitative measures evaluating the accuracy and reliability of the model's classifications compared to the reviewer assessments 168, which are treated as the ground truth. Specific performance metrics 176 calculated by the first computing device 102 may include accuracy (overall proportion of correct classifications), sensitivity (recall, or true positive rate), specificity (true negative rate), Positive Predictive Value (PPV, or precision), Negative Predictive Value (NPV), and F1-score (harmonic mean of precision and recall). The first computing device 102 may calculate the performance metrics 176 by comparing the stream of first classifications 156 generated by the first machine learning model 140 against the corresponding reviewer assessments 168 received over time for a specific criterion.

The first computing device 102 may be further configured to present the one or more determined performance metrics 176 via the user interface 132. The presentation of metrics may provide users, such as research coordinators or trial managers, with real-time feedback on the reliability of the automated screening for each eligibility requirement. Performance metrics 176 might be displayed in summary tables associated with the trial setup or dashboard 134, or visualized using charts (such as bar charts showing accuracy per criterion, trend lines showing performance over time). For example, the user interface 132 might display a table listing each criterion with its current sensitivity, specificity, and PPV based on the feedback 166 received to date. The transparency allows users to gauge confidence in the automated results and identify criteria where the model 140 may be struggling and require more careful manual oversight or more aggressive prompt refinement.

In configurations where an eligibility classification 160 is determined, the first computing device 102 may be configured to present the eligibility classification 160 for the first patient within a recruitment tracking dashboard 134 via the user interface 132. The recruitment tracking dashboard 134 may serve as a central hub for managing the patient screening and recruitment workflow. The first computing device 102 may populate the dashboard 134 with potential candidates identified by the screening process, displaying key information including their overall eligibility classification 160.

Within the recruitment tracking dashboard 134, the first computing device 102 may be configured to present a prioritized list of patients from the plurality of patients based on respective eligibility classifications 160. The dashboard 134 might display patients in a list or table format, sorted by default based on their eligibility classification 160, such as ranking patients with the highest proportional eligibility score or highest probabilistic likelihood score first. The prioritization helps research staff focus their manual review and outreach efforts on the candidates most likely to qualify for the trial.

The first computing device 102 may further be configured to enable filtering of patients presented within the recruitment tracking dashboard 134 based on user-specified parameters. The user interface 132 may provide filtering controls allowing users to narrow down the displayed patient list based on criteria such as eligibility classification 160 range (such as show only patients with >70% proportional score), the classification 156 for a specific eligibility requirement (such as show patients who met the ‘diagnosis’ criterion but were uncertain for ‘prior treatment’), demographic parameters, or current recruitment status (such as show all patients ‘Contacted Interested’).

Additionally, the first computing device 102 may be configured to enable the addition of notes associated with the first patient within the recruitment tracking dashboard 134. The dashboard 134 user interface 132 may include a feature allowing users to enter and view free-text notes for each patient, facilitating communication among the research team and tracking specific interactions, scheduling details, or reasons for ineligibility not captured by structured status fields.

In certain advanced implementations, the first computing device 102 may be configured to initiate an automated communication to the first patient based on an interaction with the recruitment tracking dashboard 134. For example, the dashboard 134 might include a button or action associated with a potentially eligible patient (‘Initiate Contact’). Upon activation by an authorized user, the first computing device 102 could trigger the sending of a predefined message template through a secure communication channel, such as the institution's patient portal (such as Epic MyChart®) or secure email. Message templates might require customization or approval before sending, potentially incorporating details about the specific clinical trial.

The system 100 may be implemented using various configurations of computing devices. The first computing device 102, second computing device 104, and third computing device 103 may represent distinct physical or virtual machines, such as servers, desktop computers, laptops, or cloud computing instances. For example, the third computing device 103 may be a user terminal (e.g., a workstation used by a clinical research coordinator), while the first computing device 102 acts as a web server hosting the user interface 132, and the second computing device 104 may represent one or more backend servers or cloud resources performing data processing and machine learning model execution. In alternative implementations, the functionalities of the first computing device 102 and the second computing device 104 may be consolidated onto a single computing device. Conversely, the operations described as performed by the second computing device 104 may be distributed across multiple computing devices, such as dedicated to specific tasks like machine learning inference or database management. Furthermore, while FIG. 1 depicts machine learning models 140, 142, 144, 146 associated with the second computing device 104, in certain implementations, one or more of these models might be executed partially or entirely on the first computing device 102 or even the third computing device 103, depending on the model complexity and available resources. The depicted architecture represents one possible arrangement, and the actual implementation may vary based on performance requirements, scalability needs, and deployment context.

Additionally, the present disclosure describes one or more machine learning models, such as the first machine learning model 140, second machine learning model 142, third machine learning model 144, and fourth machine learning model 146. A machine learning model may refer to a computational system trained on data to perform specific tasks, such as classification, regression, generation, or pattern recognition. In certain implementations, one or more of the machine learning models 140, 142, 144, 146 may be implemented as Large Language Models (LLMs), such as models based on the transformer architecture (e.g., GPT-series models, Gemini, Claude). However, other types of machine learning models may also be employed. For example, classification tasks (like determining the first classification 156) may additionally or alternatively use models like Support Vector Machines (SVMs), Random Forests, Naive Bayes classifiers, or specialized neural networks (e.g., Convolutional Neural Networks or Recurrent Neural Networks adapted for text). Models used for determining probabilistic scores may include logistic regression or gradient boosting machines. Models for query generation may leverage sequence-to-sequence architectures or other generative approaches beyond standard LLMs. It is also contemplated that certain operations described as using distinct models (e.g., 140, 142, 144, 146) might be performed by a single, multi-functional machine learning model. Furthermore, one or more of the described machine learning models may be optional in certain implementations. For instance, automated prompt generation via the second machine learning model 142 might be bypassed if prompts are manually created or retrieved from the library 112 and/or if the prompt generation is performed by the first machine learning model 14. These models may be executed locally on the first computing device 102 or second computing device 104, or they may be accessed remotely via APIs provided by third-party services.

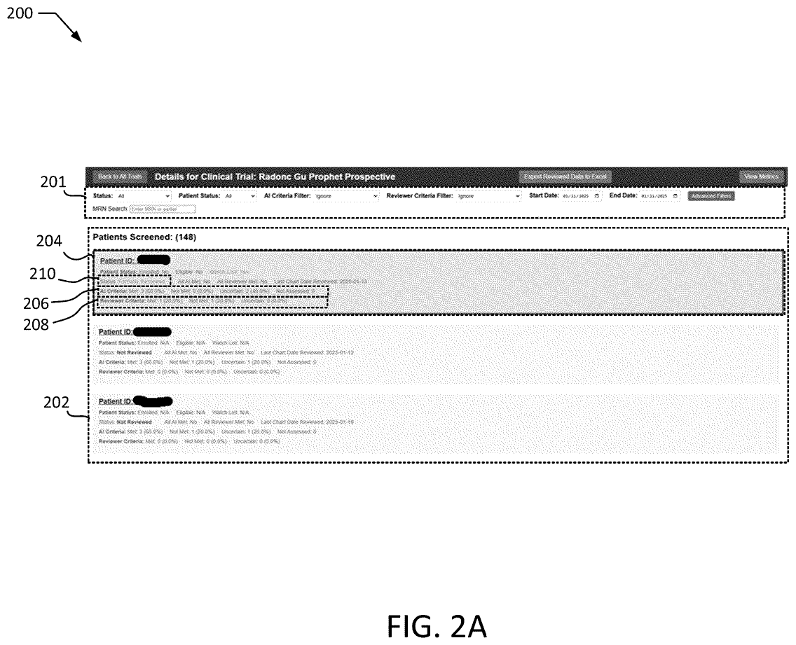

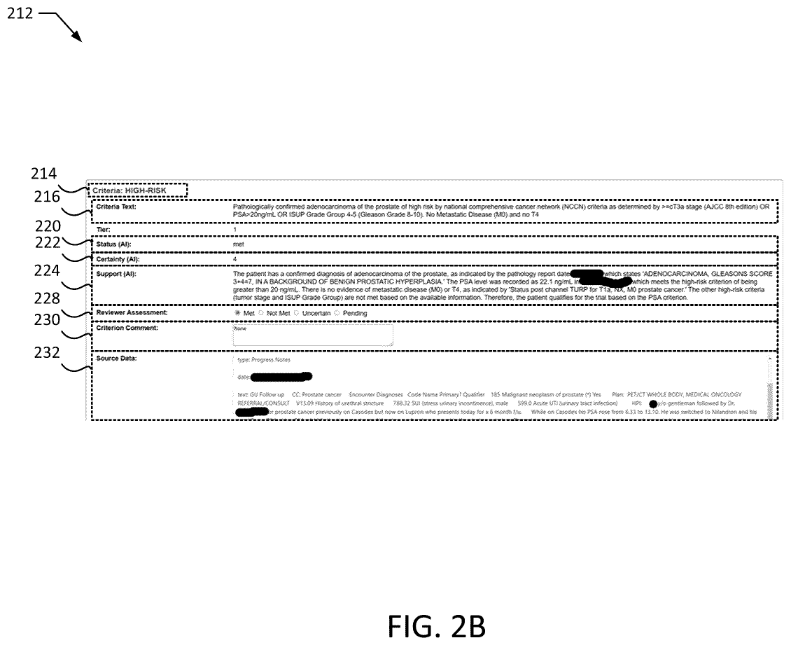

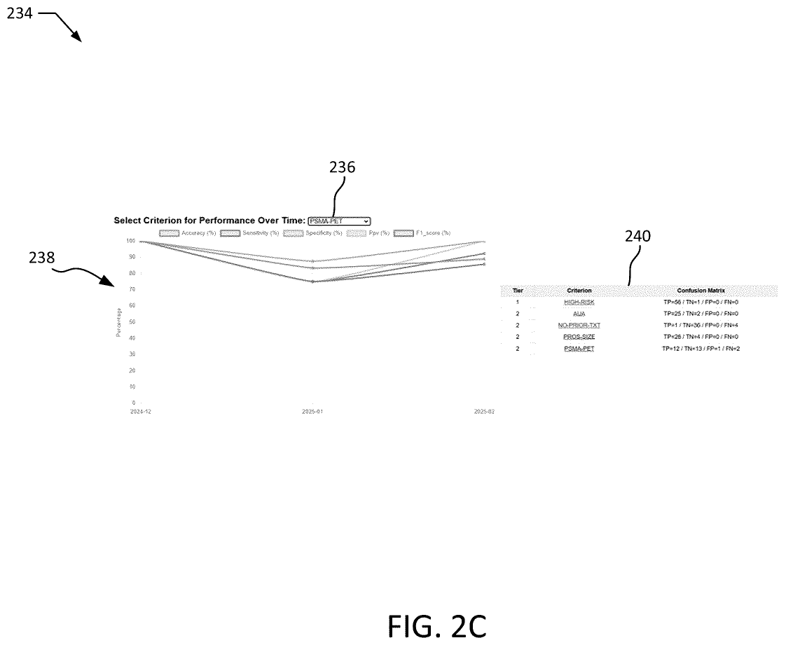

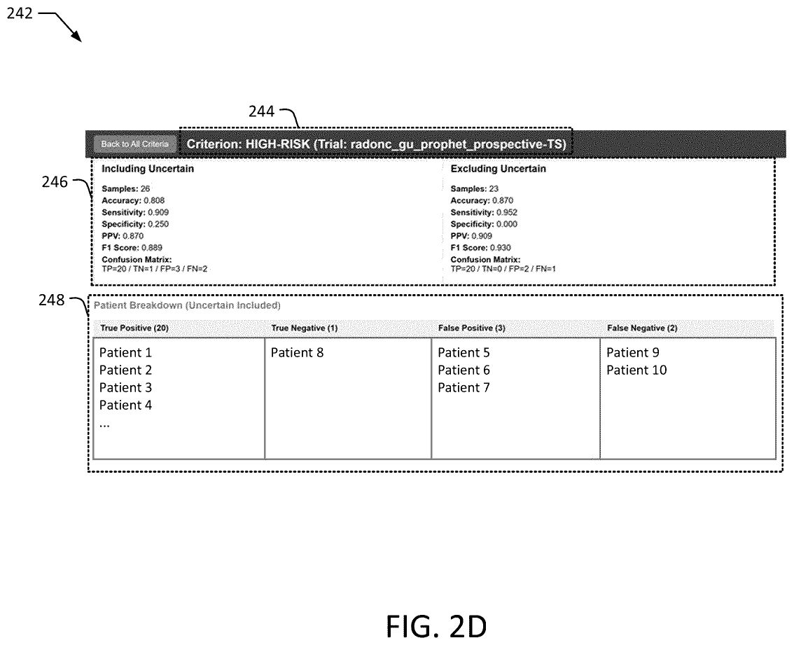

Particular implementations are discussed further herein. For example, FIG. 2A illustrates an example user interface 200, which may correspond to the user interface 132 presented by the first computing device 102 via a third computing device 103, providing a view of patients screened for a specific clinical trial. This interface 200 may function as part of the recruitment tracking dashboard 134, serving as a central location for users, such as clinical research staff, to monitor the output of the automated screening process, review patient eligibility summaries, and manage the workflow for a selected trial. The interface 200 provides tools for navigating and filtering the list of screened patients and accessing summary information derived from the classification process.

The user interface 200 may include a filter and control area 201 located towards the top of the view. This area 201 may contain various interactive elements allowing a user to refine the list of patients displayed below. For example, the filter and control area 201 may include dropdown menus to filter patients based on overall status, patient-specific status (such as recruitment status indicated by 210), criteria classifications determined by the first machine learning model 140 (e.g., AI Criteria Filter), or criteria classifications determined by human reviewers (e.g., Reviewer Criteria Filter). Additionally, the area 201 may include fields for searching by Medical Record Number (e.g., MRN) and selecting a date range to filter the patient list. An option to access advanced filtering controls may also be present within the filter and control area 201.

Below the filter and control area 201, the user interface 200 may present a patient list display 202. This display area 202 lists the individual patients who have been processed by the screening system 100 according to the current filter settings defined in area 201. The patient list display 202 may indicate the total number of patients currently being shown. The display 202 consists of multiple individual patient record displays 204, each representing one patient from the screened cohort.

Each individual patient record display 204 provides summary information pertinent to that specific patient's screening status for the selected clinical trial. The display 204 may include a patient identifier (partially obscured for privacy in the figure). Within the display 204, an AI classification summary display 206 presents aggregated results from the first machine learning model 140, potentially showing the count and percentage of eligibility requirements classified as ‘Met’, ‘Not Met’, or ‘Uncertain’. Similarly, a reviewer classification summary display 208 may present aggregated results based on human reviewer assessments 168 entered via the system, showing counts and percentages for criteria marked as ‘Met’ or ‘Not Met’ by the reviewer. Furthermore, a recruitment status indicator 210 within the display 204 may show the current stage of the patient in the review or recruitment process, such as ‘Not Reviewed’ or ‘Partially Reviewed’. Additional summary details like enrollment status or the date of the last review may also be presented within the individual patient record display 204.

FIG. 2B depicts an example user interface 212, which may be presented within the broader user interface 132, 200, potentially upon selecting a specific criterion for a patient from the patient list display 202 shown in FIG. 2A. This user interface 212 allows a user, such as a reviewer, to examine the automated assessment performed by the first machine learning model 140 for a single eligibility requirement for a particular patient. The primary function of the user interface 212 may be to present the model's findings, including its classification, certainty, and supporting rationale, alongside the source data used, and to provide interactive elements for the reviewer to input their own assessment and comments.

The upper portion of the user interface 212 may include elements identifying the specific eligibility requirement being examined. A criterion identifier display 214 indicates the name or label assigned to the requirement (e.g., “HIGH-RISK”). Below the identifier, a criterion text display 216 presents the full, detailed definition of the eligibility requirement as specified for the clinical trial. This ensures the reviewer has the precise definition readily available while evaluating the assessment. Other metadata, such as a tier assignment for the criterion, may also be displayed.

An assessment display area presents the results determined by the first machine learning model 140. An AI classification display 220 shows the first classification 156 assigned by the model (e.g., “met”). An AI certainty display 222 shows the corresponding first certainty measure 158 determined by the model (e.g., represented numerically as “4” in this example, though other scales like probabilities or qualitative labels may be used). An AI support/rationale display 224 provides textual explanation generated by the model, outlining the reasoning or evidence from the patient data 108 that led to the determined classification 156 and certainty 158.

Below the AI assessment, a reviewer feedback area provides the mechanism for capturing the second user input 166. A reviewer assessment input element 228 allows the user to select their own assessment 168 regarding whether the patient meets the criterion, typically using interactive controls like radio buttons corresponding to possible classifications (e.g., ‘Met’, ‘Not Met’, ‘Uncertain’, ‘Pending’). Adjacent to or below the assessment input 228, a reviewer comment input element 230, such as a text box, allows the reviewer to enter free-text comments, elaborating on their assessment 168 or noting specific points of agreement or disagreement with the AI's findings.

Finally, the user interface 212 may include a source data display area 232. This area presents the actual text or data from the one or more relevant data segments 154 retrieved from the patient data store 106 and provided to the first machine learning model 140 for making the classification 156. Displaying the source data allows the reviewer to directly inspect the evidence considered by the model, aiding in their assessment of the model's accuracy and rationale. Information about the source document type (e.g., “Progress Notes”) and date may also be included in this area 232.

FIG. 2C shows an example user interface 234 for displaying real-time performance data analytics related to the automated clinical trial screening process. This interface 234 may be accessible, for example, via the user interface 200 in FIG. 2A and allows users to monitor the performance metrics 176 determined by the first computing device 102 based on reviewer feedback 166. The interface 234 provides visualizations and summaries of how accurately the first machine learning model 140 is classifying specific eligibility requirements.

The user interface 234 may include interactive elements, such as a criterion selection element 236 (e.g., a dropdown menu), allowing the user to choose a specific eligibility requirement for detailed performance analysis. Based on the selection, a performance metrics plot 238 may be displayed. The plot 238 can visualize trends over time for various performance metrics 176, such as Accuracy, Sensitivity, Specificity, Positive Predictive Value (PPV), and F1-score, typically represented as percentages. Additionally, the user interface 234 may present a performance metrics table 240. The table 240 can provide a summary for multiple criteria simultaneously, potentially showing tier information, criterion names, and confusion matrix data (e.g., counts of True Positives (TP), True Negatives (TN), False Positives (FP), and False Negatives (FN)) derived from comparing model classifications 156 with reviewer assessments 168.

FIG. 2D presents an example user interface 242 offering a more detailed view of the performance metrics 176 for a single, specific eligibility requirement. This interface 242 may be accessed, for instance, by selecting a criterion from the performance metrics table 240 shown in FIG. 2C or through other navigation elements within the overall user interface 132, 200. The purpose of this interface 242 may be to provide an in-depth analysis of the first machine learning model's 140 performance on a chosen criterion, allowing users to understand the nature of any classification errors.

The user interface 242 features a criterion identifier display 244, which specifies the eligibility requirement (e.g., “HIGH-RISK”) and the associated clinical trial context. Below this, one or more performance metrics summaries 246 are displayed. These summaries 246 present calculated performance metrics 176 based on comparing the first classifications 156 with the reviewer assessments 168. Metrics shown may include the total number of samples reviewed, Accuracy, Sensitivity, Specificity, Positive Predictive Value (PPV), F1 Score, and the raw counts from the confusion matrix (True Positives, True Negatives, False Positives, False Negatives). In the depicted example, two summaries 246 are shown side-by-side and may performance metrics calculated under different conditions (e.g., including versus excluding patients where the model 140 generated an ‘Uncertain’ classification).

The lower portion of the user interface 242 includes a patient list breakdown 248. This section categorizes the individual patients reviewed for the selected criterion based on their outcome in the confusion matrix analysis (True Positive, True Negative, False Positive, False Negative). Under each category heading, the interface 248 lists the specific identifiers (partially obscured in the figure) of the patients falling into that category. This breakdown allows users to directly identify which patients were correctly classified and, more importantly, which patients represent specific types of classification errors (False Positives and False Negatives), facilitating targeted review or analysis of potentially problematic cases.

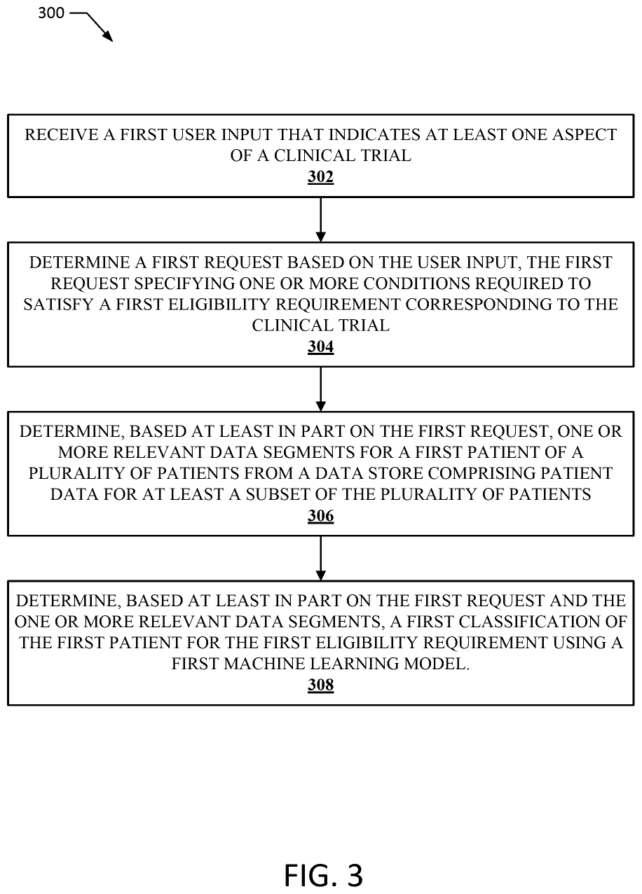

FIG. 3 depicts a method 300 for clinical trial eligibility evaluation according to one aspect of the present disclosure. The method 300 may be implemented on a computer system, such as the system 100. For example, the method 300 may be implemented by the computing device 102. The method 300 may also be implemented by a set of instructions stored on a computer readable medium that, when executed by a processor, cause the computing device to perform the method 300. Although the examples below are described with reference to the flowchart illustrated in FIG. 3, many other methods of performing the acts associated with FIG. 3 may be used. For example, the order of some of the blocks may be changed, certain blocks may be combined with other blocks, one or more of the blocks may be repeated, and some of the blocks may be optional.

At block 302, the method 300 may include receiving a user input that indicates at least one aspect of a clinical trial. For example, the first computing device 102 may receive a user input 150 that indicates at least one aspect of a clinical trial via a user interface 132. In certain implementations, the at least one aspect of the clinical trial indicated by the user input 150 may include the first eligibility requirement itself. An eligibility requirement may refer to a specific condition or characteristic that a patient must meet (or not meet) to qualify for participation in the clinical trial. For instance, a user might directly input a specific eligibility requirement like “ECOG performance status 0-2” via the user interface 132. In other implementations, the at least one aspect of the clinical trial indicated by the user input 150 may include at least one of a medical condition (e.g., ‘Stage IV Prostate Cancer’), a diagnostic category (e.g., ‘Oncology’), a therapeutic area (e.g., ‘Cardiology’), a patient demographic (e.g., ‘Age>=18 years’), or a combination thereof. Receiving these broader aspects may guide subsequent operations performed by the first computing device 102 or the second computing device 104.

At block 304, the method 300 may include determining a first request based on the user input. For example, the first computing device 102 may determine a first request 152 based on the received user input 150. The first request 152 may specify one or more conditions required to satisfy a first eligibility requirement corresponding to the clinical trial. A request may refer to a structured or unstructured query, prompt, set of instructions used to guide data retrieval or model execution, or a combination thereof. Conditions may refer to the specific logical, textual, or numerical constraints defining the eligibility requirement. In certain implementations, the first machine learning model 140 may be a large language model (LLM). In such cases, the first request 152 determined by the first computing device 102 may include a prompt configured for execution by the first machine learning model 140. For example, the prompt might instruct the first machine learning model 140 to assess if a patient meets a specific criterion based on provided text. In some implementations, determining the first request 152 may include the first computing device 102 determining the prompt using the first machine learning model 140, a second machine learning model 142, or a combination thereof, based on the user input 150. For example, the second machine learning model 142, potentially hosted on the second computing device 104, may automatically generate the prompt text based on the received user input 150. In certain implementations, the first computing device 102 or the second computing device 104 may determine one or more external data sources 120 based on the user input 150, potentially guided by the second machine learning model 142. The first computing device 102 may then query the one or more external data sources 120. The determination of the first request 152 by the first computing device 102 may be further based on results from querying the one or more external data sources 120. For instance, definitions or criteria retrieved from external sources 120 may be integrated into the first request 152. In certain implementations, the one or more external data sources 120 may include at least one of a public medical literature database (e.g., PubMed), a clinical trial registry (e.g., ClinicalTrials. gov), or a curated knowledge base (e.g., UMLS, SNOMED-CT).

In certain implementations, prior to determining relevant data segments, the method 300 may include selecting the plurality of patients. For example, the first computing device 102 may select the plurality of patients based at least in part on the user input 150, the first request 152, or a combination thereof. This pre-selection step may narrow down a larger patient population data 118 stored in the data store 106 to a relevant cohort for detailed screening. In some implementations, selecting the plurality of patients may include determining, using a third machine learning model 146 based on the user input 150, the first request 152, or a combination thereof, one or more structured data queries 164. For example, the third machine learning model 146, potentially hosted on the second computing device 104, may translate criteria into SQL queries. The selection may further include the first computing device 102 executing the one or more structured data queries 164 against a larger patient population data 118 within the data store 106.

At block 306, the method 300 may include determining one or more relevant data segments for a first patient of a plurality of patients from a data store including patient data for at least a subset of the plurality of patients. For example, the first computing device 102 may determine, based at least in part on the first request 152, one or more relevant data segments 154 for a first patient selected from the plurality of patients (potentially pre-selected as described above) from a data store 106 including patient data 108. The patient data 108 within the data store 106 may include various types of information, such as structured data (e.g., lab results) and unstructured text data (e.g., clinical notes, pathology reports) from electronic health record (EHR) systems. Determining the relevant data segments 154 may involve techniques like vector-based similarity search (e.g., Retrieval-Augmented Generation or RAG) using vector embeddings 110 derived from the patient data 108, potentially orchestrated by the first computing device 102 using frameworks like LlamaIndex.

At block 308, the method 300 may include determining a first classification of the first patient for the first eligibility requirement using a first machine learning model. For example, the first computing device 102 may determine, based at least in part on the first request 152 and the one or more relevant data segments 154, a first classification 156 of the first patient for the first eligibility requirement using a first machine learning model 140, which may be hosted on or accessed via the second computing device 104. The first classification 156 may indicate whether the patient meets the requirement (e.g., ‘Met’, ‘Not Met’, ‘Uncertain’). In certain implementations, the method 300 may further include determining, potentially by the first machine learning model 140 or the first computing device 102, a first certainty measure 158 associated with the first classification 156. For example, the first computing device 102 may determine a first certainty measure 158 indicating the confidence (e.g., 0.95) in the determined first classification 156. In some implementations, the method 300 may further include repeating the step of determining a first classification 156 for each patient of at least a second subset of the plurality of patients. For example, the first computing device 102 may iteratively determine classifications 156 for multiple patients in the selected cohort.

In certain implementations, the method 300 may include determining an overall eligibility classification for the patient. For example, after determining the first classification 156 for the first patient (potentially for one or multiple eligibility requirements), the first computing device 102 may determine an eligibility classification 160 of the first patient for the clinical trial based on the determined first classification(s) 156. In cases involving multiple eligibility requirements, the method 300 may include repeating the steps of determining relevant data segments 154 and determining the first classification 156 for each eligibility requirement of a plurality of eligibility requirements corresponding to the clinical trial prior to determining the eligibility classification 160 of the first patient. The eligibility classification 160 may then be determined by the first computing device 102 based on the classifications 156 determined for the plurality of eligibility requirements. For instance, the eligibility classification 160 may indicate a proportion of the plurality of eligibility requirements for which the determined classification 156 indicates the requirement is met (e.g., 8 out of 10 criteria met, resulting in an 80% proportional score). This proportional score might be calculated by the first computing device 102 or potentially by a dedicated machine learning model like the fourth machine learning model 144.

In some implementations, the method 300 may include receiving feedback and refining the requests. For example, the first computing device 102 may receive a second user input 166 via a user interface 132, where the second user input 166 indicates a reviewer assessment 168 of the first classification 156 for the first patient. The reviewer assessment 168 may be provided by a human expert evaluating the model's output. The method 300 may further include determining that a discrepancy 170 exists between the first classification 156 and the reviewer assessment 168 indicated by the second user input 166. For example, the first computing device 102 may compare the model's output 156 with the reviewer's input 168. Based at least in part on the discrepancy 170, the method 300 may include determining an adjustment 172 applicable to the first request 152. For example, the first computing device 102, potentially using the second machine learning model 142, may determine an adjustment 172 based on the discrepancy 170, the second user input 166 (including comments), and the first request 152. The method 300 may also include determining a second request 174 by applying the adjustment 172 to the first request 152. For example, the first computing device 102 may modify the first request 152 according to the determined adjustment 172 to generate the second request 174. In certain implementations, the method 300 may further include receiving a third user input 178 indicating a recruitment outcome for the first patient in the clinical trial (e.g., ‘Successfully Enrolled’, ‘Screen Failed’). The adjustment 172 may be determined based further on the third user input 178, allowing long-term outcomes stored as tracking data 116 to inform request refinement. The refined second request 174 may then be used by the first computing device 102 for subsequent classifications or stored in the request library 112.

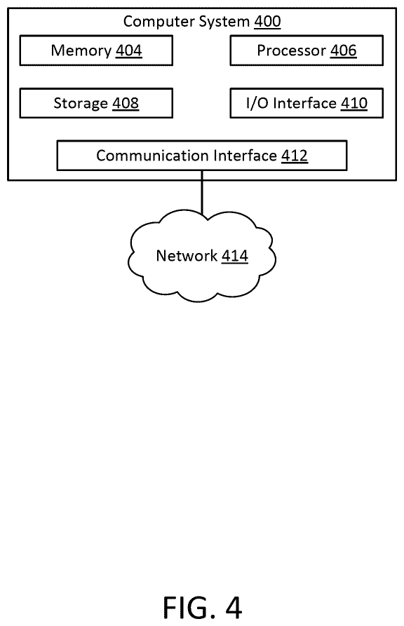

FIG. 4 illustrates an example computer system 400 that may be utilized to implement one or more of the devices and/or components discussed herein, such as the first computing device 102, second computing device 104, and/or third computing device 103 involved in the clinical trial screening system. In particular embodiments, one or more computer systems 400 perform one or more steps of one or more methods described or illustrated herein, such as steps related to AI-augmented clinical trial screening. In particular embodiments, one or more computer systems 400 provide the functionalities described or illustrated herein. In particular embodiments, software running on one or more computer systems 400 performs one or more steps of one or more methods described or illustrated herein or provides the functionalities described or illustrated herein. Particular embodiments include one or more portions of one or more computer systems 400. Herein, a reference to a computer system may encompass a computing device, and vice versa, where appropriate. Moreover, a reference to a computer system may encompass one or more computer systems, where appropriate.

This disclosure contemplates any suitable number of computer systems 400. This disclosure contemplates the computer system 400 taking any suitable physical form. As example and not by way of limitation, the computer system 400 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the computer system 400 may include one or more computer systems 400; be unitary or distributed (e.g., distributing user interface, data processing, and AI model execution components across devices 102, 103, 104); span multiple locations; span multiple machines; span multiple data centers; or reside in a cloud, which may include one or more cloud components in one or more networks (e.g., utilizing cloud resources for AI processing or data storage). Where appropriate, one or more computer systems 400 may perform without substantial spatial or temporal limitation one or more steps of one or more methods described or illustrated herein. As an example and not by way of limitation, one or more computer systems 400 may perform in real time or in batch mode one or more steps of one or more methods described or illustrated herein (e.g., performing screening tasks). One or more computer systems 400 may perform at different times or at different locations one or more steps of one or more methods described or illustrated herein, where appropriate.

In particular embodiments, computer system 400 includes a processor 406, memory 404, storage 408, an input/output (I/O) interface 410, and a communication interface 412. Although this disclosure describes and illustrates a particular computer system having a particular number of particular components in a particular arrangement, this disclosure contemplates any suitable computer system having any suitable number of any suitable components in any suitable arrangement.

In particular embodiments, the processor 406 includes hardware for executing instructions, such as those making up a computer program. As an example and not by way of limitation, to execute instructions, the processor 406 may retrieve (or fetch) the instructions from an internal register, an internal cache, memory 404, or storage 408; decode and execute the instructions (e.g., instructions implementing the described screening functionalities); and then write one or more results to an internal register, internal cache, memory 404, or storage 408. In particular embodiments, the processor 406 may include one or more internal caches for data, instructions, or addresses. This disclosure contemplates the processor 406 including any suitable number of any suitable internal caches, where appropriate. As an example and not by way of limitation, the processor 406 may include one or more instruction caches, one or more data caches, and one or more translation lookaside buffers (TLBs). Instructions in the instruction caches may be copies of instructions in memory 404 or storage 408, and the instruction caches may speed up retrieval of those instructions by the processor 406. Data in the data caches may be copies of data in memory 404 or storage 408 that are to be operated on by computer instructions; the results of previous instructions executed by the processor 406 that are accessible to subsequent instructions or for writing to memory 404 or storage 408; or any other suitable data. The data caches may speed up read or write operations by the processor 406. The TLBs may speed up virtual-address translation for the processor 406. In particular embodiments, processor 406 may include one or more internal registers for data, instructions, or addresses. This disclosure contemplates the processor 406 including any suitable number of any suitable internal registers, where appropriate. Where appropriate, the processor 406 may include one or more arithmetic logic units (ALUs), be a multi-core processor, include specialized processing units (e.g., GPUs or TPUs) suitable for accelerating AI computations, or include one or more processors 406. Although this disclosure describes and illustrates a particular processor, this disclosure contemplates any suitable processor.

In particular embodiments, the memory 404 includes main memory for storing instructions for the processor 406 to execute or data for processor 406 to operate on. As an example, and not by way of limitation, computer system 400 may load instructions from storage 408 or another source (such as another computer system 400) to the memory 404. The processor 406 may then load the instructions from the memory 404 to an internal register or internal cache. To execute the instructions, the processor 406 may retrieve the instructions from the internal register or internal cache and decode them. During or after execution of the instructions, the processor 406 may write one or more results (which may be intermediate or final results) to the internal register or internal cache. The processor 406 may then write one or more of those results to the memory 404. Memory 404 may temporarily hold data such as patient information segments or AI model parameters during processing. In particular embodiments, the processor 406 executes only instructions in one or more internal registers or internal caches or in memory 404 (as opposed to storage 408 or elsewhere) and operates only on data in one or more internal registers or internal caches or in memory 404 (as opposed to storage 408 or elsewhere). One or more memory buses (which may each include an address bus and a data bus) may couple the processor 406 to the memory 404. The bus may include one or more memory buses, as described in further detail below. In particular embodiments, one or more memory management units (MMUs) reside between the processor 406 and memory 404 and facilitate accesses to the memory 404 requested by the processor 406. In particular embodiments, the memory 404 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 404 may include one or more memories 404, where appropriate. Although this disclosure describes and illustrates particular memory implementations, this disclosure contemplates any suitable memory implementation.

In particular embodiments, the storage 408 includes mass storage for data or instructions. As an example and not by way of limitation, the storage 408 may include a hard disk drive (HDD), a floppy disk drive, flash memory, an optical disc, a magneto-optical disc, magnetic tape, or a Universal Serial Bus (USB) drive or a combination of two or more of these. Storage 408 may persistently store patient data (e.g., EHR data 108), vector embeddings 110, AI models (e.g., 140, 142, 144, 146), stored requests 114, and software instructions related to the clinical trial screening system. The storage 408 may include removable or non-removable (or fixed) media, where appropriate. The storage 408 may be internal or external to computer system 400, where appropriate. In particular embodiments, the storage 408 is non-volatile, solid-state memory. In particular embodiments, the storage 408 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 408 taking any suitable physical form. The storage 408 may include one or more storage control units facilitating communication between processor 406 and storage 408, where appropriate. Where appropriate, the storage 408 may include one or more storages 408. Although this disclosure describes and illustrates particular storage, this disclosure contemplates any suitable storage.

In particular embodiments, the I/O Interface 410 includes hardware, software, or both, providing one or more interfaces for communication between computer system 400 and one or more I/O devices. The computer system 400 may include one or more of these I/O devices, where appropriate. One or more of these I/O devices may enable communication between a person (i.e., a user) and computer system 400. As an example and not by way of limitation, an I/O device may include a keyboard, keypad, microphone, monitor, screen, display panel (e.g., for presenting the user interface 132 for inputting trial aspects or reviewing screening results), mouse, printer, scanner, speaker, still camera, stylus, tablet, touch screen (e.g., for receiving user inputs 150, 166, 178), trackball, video camera, another suitable I/O device or a combination of two or more of these. An I/O device may include one or more sensors. Where appropriate, the I/O Interface 410 may include one or more device or software drivers enabling processor 406 to drive one or more of these I/O devices. The I/O interface 410 may include one or more I/O interfaces 410, where appropriate. Although this disclosure describes and illustrates a particular I/O interface, this disclosure contemplates any suitable I/O interface or combination of I/O interfaces.

In particular embodiments, communication interface 412 includes hardware, software, or both providing one or more interfaces for communication (such as, for example, packet-based communication) between computer system 400 and one or more other computer systems 400 (e.g., between devices 102, 103, 104) or one or more networks 414. As an example and not by way of limitation, communication interface 412 may include a network interface controller (NIC) or network adapter for communicating with an Ethernet or any other wire-based network or a wireless NIC (WNIC) or wireless adapter for communicating with a wireless network, such as a Wi-Fi network. This interface enables communication with EHR systems, external data sources 120, remote AI model APIs, or data stores 106, potentially requiring secure protocols (e.g., HTTPS) due to the sensitivity of patient data. This disclosure contemplates any suitable network 414 and any suitable communication interface 412 for the network 414. As an example and not by way of limitation, the network 414 may include one or more of an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 may communicate with a wireless PAN (WPAN) (such as, for example, a Bluetooth® WPAN), a WI-FI network, a WI-MAX network, a cellular telephone network (such as, for example, a Global System for Mobile Communications (GSM) network), or any other suitable wireless network or a combination of two or more of these. Computer system 400 may include any suitable communication interface 412 for any of these networks, where appropriate. Communication interface 412 may include one or more communication interfaces 412, where appropriate. Although this disclosure describes and illustrates a particular communication interface implementations, this disclosure contemplates any suitable communication interface implementation.

The computer system 400 may also include a bus 402. The bus 402 may include hardware, software, or both and may communicatively couple the components of the computer system 400 to each other. As an example and not by way of limitation, the bus 402 may include an Accelerated Graphics Port (AGP) or any other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-PIN-count (LPC) bus, a memory bus, a Micro Channel Architecture (MCA) bus, a Peripheral Component Interconnect (PCI) bus, a PCI-Express (PCIe) bus (useful for high-speed components like GPUs or SSDs), a serial advanced technology attachment (SATA) bus, a Video Electronics Standards Association local bus (VLB), or another suitable bus or a combination of two or more of these buses. The bus 402 may include one or more buses, where appropriate. Although this disclosure describes and illustrates a particular bus, this disclosure contemplates any suitable bus or interconnect.

Herein, a computer-readable non-transitory storage medium or media may include one or more semiconductor-based or other types of integrated circuits (ICs) (e.g., field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. Such media may store instructions for performing the AI-augmented clinical trial screening methods described herein. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, features, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A method comprising:
receiving a first user input that indicates at least one aspect of a clinical trial;
determining a first request based on the user input, the first request specifying one or more conditions required to satisfy a first eligibility requirement corresponding to the clinical trial;
determining, based at least in part on the first request, one or more relevant data segments for a first patient of a plurality of patients from a data store comprising patient data for at least a subset of the plurality of patients; and
determining, based at least in part on the first request and the one or more relevant data segments, a first classification of the first patient for the first eligibility requirement using a first machine learning model.

2. The method of claim 1,
wherein,
the at least one aspect of the clinical trial indicated by the user input includes the first eligibility requirement.

3. The method of claim 1,
wherein,
the at least one aspect of the clinical trial indicated by the user input includes at least one of a medical condition, a diagnostic category, a therapeutic area, a patient demographic, or a combination thereof.

4. The method of claim 1,
wherein,
the first machine learning model is a large language model, and
the first request includes a prompt configured for execution by the first machine learning model.

5. The method of claim 4,
wherein,
the determining of the first request includes determining, based on the user input, the prompt using the first machine learning model, a second machine learning model, or a combination thereof.

6. The method of claim 5, further comprising:
determining one or more external data sources based on the user input; and
querying the one or more external data sources,
wherein,
the determining of the first request is further based on results from the querying of the one or more external data sources.

7. The method of claim 6,
wherein,
the one or more external data sources includes at least one of a public medical literature database, a clinical trial registry, or a curated knowledge base.

8. The method of claim 1, further comprising:
determining, by the first machine learning model, a first certainty measure associated with the first classification.

9. The method of claim 1, further comprising:
repeating the step of determining a first classification for each patient of at least a second subset of the plurality of patients.

10. The method of claim 1, further comprising:
determining, based on the first classification for the first patient, an eligibility classification of the first patient for the clinical trial.

11. The method of claim 10, further comprising:
repeating the steps of the determining of the one or more relevant data segments and the determining of the first classification for each eligibility requirement of a plurality of eligibility requirements corresponding to the clinical trial prior to determining the eligibility classification of the first patient,
wherein,
the eligibility classification is determined based on the classifications determined for the plurality of eligibility requirements.

12. The method of claim 11,
wherein,
the eligibility classification indicates a proportion of the plurality of eligibility requirements for which the determined classification indicates the requirement is met.

13. The method of claim 1, further comprising:
selecting the plurality of patients based at least in part on the user input, the first request, or a combination thereof prior to the determining of the one or more relevant data segments.

14. The method of claim 13,
wherein,
the selecting of the plurality of patients includes:
determining, using a machine learning model based on the user input, the first request, or a combination thereof, one or more structured data queries, and
executing the one or more structured data queries against a larger patient population including the plurality of patients.

15. The method of claim 1, further comprising:
receiving a second user input via a user interface, the second user input indicating a reviewer assessment of the first classification for the first patient.

16. The method of claim 15, further comprising:
determining that a discrepancy exists between the first classification and the reviewer assessment indicated by the second user input;
determining, based at least in part on the discrepancy, an adjustment applicable to the first request based on at least one of the second user input, the first request, or a combination thereof; and
determining a second request by applying the adjustment to the first request.

17. The method of claim 16, further comprising:
receiving a third user input indicating a recruitment outcome for the first patient in the clinical trial,
wherein,
the adjustment is determined based further on the third user input.

18. A system comprising:
a processor; and
a memory storing instructions which, when executed by the processor, cause the processor to perform operations comprising:
receiving a user input that indicates at least one aspect of a clinical trial;
determining a first request based on the user input, the first request specifying one or more conditions required to satisfy a first eligibility requirement corresponding to the clinical trial;
determining, based at least in part on the first request, one or more relevant data segments for a first patient of a plurality of patients from a data store comprising patient data for at least a subset of the plurality of patients; and
determining, based at least in part on the first request and the one or more relevant data segments, a first classification of the first patient for the first eligibility requirement using a first machine learning model.

19. The system of claim 18,
wherein,
the operations further include:
receiving a second user input via a user interface, the second user input indicating a reviewer assessment of the first classification for the first patient,
determining that a discrepancy exists between the first classification and the reviewer assessment indicated by the second user input,
determining, based at least in part on the discrepancy, an adjustment applicable to the first request based on at least one of the second user input, the first request, or a combination thereof, and
determining a second request by applying the adjustment to the first request.

20. A non-transitory, computer-readable medium storing instructions which, when executed by a processor, cause the processor to perform operations, comprising:
receiving a user input that indicates at least one aspect of a clinical trial;
determining a first request based on the user input, the first request specifying one or more conditions required to satisfy a first eligibility requirement corresponding to the clinical trial;
determining, based at least in part on the first request, one or more relevant data segments for a first patient of a plurality of patients from a data store comprising patient data for at least a subset of the plurality of patients; and
determining, based at least in part on the first request and the one or more relevant data segments, a first classification of the first patient for the first eligibility requirement using a first machine learning model.
